(19) United States
(12) Patent Application Publication
(10) Pub. No.: US 2026/0363185 A1
(43) Pub. Date: Nov. 19, 2026

(54) TOMATO PLANT, METHOD FOR PRODUCING TOMATO PLANT, METHOD FOR IMPARTING PROPERTY OF LOW FRUIT pH, AND USE OF SNP MARKER

(71) Applicant: KAGOME CO., LTD., Tokyo (JP)

(72) Inventors: Yuki OI, Nasushiobara-shi (JP); Satoshi ASANO, Nasushiobara-shi (JP); Ayumu ONOZATO, Nasushiobara-shi (JP); Junji KIMBARA, Nasushiobara-shi (JP)

(73) Assignee: KAGOME CO., LTD., Tokyo (JP)

(21) Appl. No.: 19/671,148

(22) Filed: May 7, 2026

(30) Foreign Application Priority Data
May 8, 2025 (JP) 2025-078135
Apr. 10, 2026 (JP) 2026-065155

(51) Int. Cl. A01H 1/04; A01H 1/00; C12Q 1/6895

(52) U.S. Cl. CPC A01H 1/045; A01H 1/101; A01H 1/1265; C12Q 1/6895; C12Q 2600/13; C12Q 2600/158

(57) ABSTRACT

A novel tomato plant is provided. A tomato plant in accordance with one aspect of the present disclosure is homozygous for a null allele of a lycopene β-cyclase gene on chromosome 6 and is heterozygous or homozygous for a low-fruit-pH locus.

### CROSS-REFERENCE TO RELATED APPLICATIONS

This application claims priority to Japanese Application No. 2026-065155, filed Apr. 10, 2026, and Japanese Application No. 2025-078135, filed May 8, 2025, the disclosures of each of which are herein incorporated in their entireties.

### REFERENCE TO AN ELECTRONIC SEQUENCE LISTING

The content of the electronic sequence listing (247322021200.xml; Size: 9,357 bytes; and Date of Creation: May 6, 2026) is herein incorporated by reference in its entirety.

### FIELD

The present invention relates to a tomato plant, a method for producing a tomato plant, a method for imparting a property of low fruit pH, and use of a SNP marker, and more specifically, to a tomato plant, a method for producing a tomato plant, a method for imparting a property of low fruit pH to a tomato plant that produces fruit having a high lycopene content, and use of a SNP marker.

### BACKGROUND

Lycopene is a functional component that has been reported to exhibit various functions such as an antioxidant effect, and is known to be contained in large amounts in tomatoes.

Regarding lycopene content, Non-patent Literature 1 reports that when tomatoes are homozygous for an old-gold-crimson (ogc) gene, the lycopene content increases compared to normal.

Furthermore, Non-patent Literature 2 describes that a B gene, which is associated with pigmentation in tomatoes, is involved in the biosynthesis of β-carotene, and that lycopene β-cyclase encoded by the B gene is an enzyme that converts lycopene to β-carotene. Non-patent Literature 2 also states that the ogc gene has been demonstrated to be a null allele of the B gene, and that a transgenic experiment has shown that the phenotype of an individual in which the function of the B gene is suppressed matches the phenotype of the ogc gene.

Tomatoes are also frequently used as processed foods. Non-patent Literature 3 reports that while a typical fully ripe tomato fruit has a pH of 4.2 to 4.9, it is desirable for the pH to be not more than 4.3 for microbial control when the tomato is used as a processed food.

Regarding the pH of tomatoes, Non-patent Literature 4 suggests that a quantitative trait locus (QTL) involved in pH derived from cultivated tomato species is located on chromosome 6.

### CITATION LIST

[Non-patent Literature 1] Thompson et al., (1965) Proc. Amer. Soc. Hort. Sci., 86:610-616. [Non-patent Literature 2] Ronen et al., (2000), PNAS, vol. 97, no. 20, 11102-11107. [Non-patent Literature 3]“Tomato jusu rui no HACCP donyu manyuaru (manual for HACCP implementation for tomato juice products)”, pp. 43-51, (1999), Japan Tomato Processors Association. [Non-patent Literature 4] Kimbara et al., (2018), Euphytica, 214(11).

### SUMMARY

As described above, when tomatoes are used as processed foods, it is desirable that the fruit pH be not more than 4.3. However, upon investigation by the inventors of the present invention, it was confirmed that in lines possessing the ogc gene, which leads to a high lycopene content, the pH of the fruit is generally high.

Therefore, the present invention has been made in view of the above-mentioned problem, and an object of one aspect of the present invention is to provide a tomato plant that produces fruit having a high lycopene content and a low pH.

The inventors of the present invention conducted intensive studies in order to achieve the above object. As a result, the inventors of the present invention discovered a single nucleotide polymorphism (SNP) marker associated with a quantitative trait locus that is involved in pH and that is located on chromosome 6, and thereby completed the present invention.

That is, a tomato plant in accordance with one aspect of the present invention is characterized by being homozygous for a null allele of a lycopene β-cyclase gene on chromosome 6 and being heterozygous or homozygous for a low-fruit-pH locus on chromosome 6, the low-fruit-pH locus being identified by at least one SNP marker selected from the group consisting of (a) and (b) below:
(a) a SNP marker that is C at a base corresponding to a base at position 51 in a nucleotide sequence set forth in SEQ ID NO: 1; and
(b) a SNP marker that is T at a base corresponding to a base at position 51 in a nucleotide sequence set forth in SEQ ID NO: 2.

Furthermore, a method for producing a tomato plant in accordance with one aspect of the present invention, for producing the above-described tomato plant, is characterized by including: a crossing step of crossing a tomato plant having the null allele of the lycopene β-cyclase gene with a tomato plant having the low-fruit-pH locus; and a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus.

Furthermore, a method for imparting a property of low fruit pH to a tomato plant that produces fruit having a high lycopene content in accordance with one aspect of the present invention is characterized by including a crossing step of crossing a tomato plant having a null allele of a lycopene β-cyclase gene on chromosome 6 with a tomato plant having a low-fruit-pH locus on chromosome 6; and a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus.

Furthermore, use of a SNP marker for detecting a low-fruit-pH locus that results in low-pH tomato fruit, in accordance with one aspect of the present invention, is characterized by including detecting at least one SNP marker selected from the group consisting of (a) and (b) below:
(a) a SNP marker that is C at a base corresponding to a base at position 51 in a nucleotide sequence set forth in SEQ ID NO: 1; and
(b) a SNP marker that is T at a base corresponding to a base at position 51 in a nucleotide sequence set forth in SEQ ID NO: 2.

According to one aspect of the present invention, it is possible to provide a tomato plant that produces fruit having a high lycopene content and a low pH.

### BRIEF DESCRIPTION OF THE FIGURES

FIG. 1 shows a comparison of the pH values of the respective fruits of F2 individuals obtained by crossing a line with low tomato fruit pH (PK461) and a line with high tomato fruit pH (PK367).

FIG. 2 shows the nucleotide sequences of PCR-amplified fragments containing bases of pH-related SNP markers prepared in Example 2.

### DETAILED DESCRIPTION

An aspect of the present invention is described below, but the present invention is not limited thereto. In the present specification, “A to B”, unless otherwise specified, indicates a range of not less than A and not more than B.

### Tomato Plant

A tomato plant in accordance with one aspect of the present invention is a tomato plant that is homozygous for a null allele of a lycopene β-cyclase gene on chromosome 6 and that has a low-fruit-pH locus.

In the present specification, a “tomato plant” is a plant belonging to Solanum lycopersicum.

In the present specification, a “plant” may be a plant body or a part of a plant body. In the present specification, a “plant body” refers to a plant individual representing an entire plant. Examples of a part of a plant body include an organ, a tissue, and a cell. Examples of an organ include a petal, a corolla, a flower, a leaf, a seed, a fruit, a stem, and a root. A tissue is, for example, a part of an organ.

### Lycopene β-Cyclase Gene

The tomato plant in accordance with the present aspect is homozygous for the null allele of the lycopene β-cyclase gene on chromosome 6.

Lycopene β-cyclase is an enzyme that converts lycopene, which is a type of carotenoid contained in the fruit of a tomato plant, into β-carotene. The lycopene β-cyclase gene is a gene that encodes lycopene β-cyclase and is a dominant gene whose trait is expressed when the genotype is homozygous or heterozygous.

In the present specification, the “null allele of the lycopene β-cyclase gene” is a loss-of-function allele of the lycopene β-cyclase gene. When a tomato plant is homozygous for the null allele of the lycopene β-cyclase gene, the phenotype of the null allele is manifested in the fruit. Therefore, the null allele of the lycopene β-cyclase gene can also be said to be a recessive gene. The null allele of the lycopene β-cyclase gene has, in the lycopene β-cyclase gene, a mutation that prevents the function of lycopene β-cyclase from being exhibited, and as a result, lycopene is not converted into β-carotene. Accordingly, in a tomato plant that is homozygous for the null allele of the lycopene β-cyclase gene, the lycopene content in the fruit is higher than that in a tomato plant having a genotype that allows the lycopene β-cyclase gene to be normally expressed. Note that it is considered as a result of genomic nucleotide sequence analysis that there are three homologs of the lycopene β-cyclase gene in a tomato plant, located one each on chromosome 4, chromosome 6, and chromosome 10. Among these, the lycopene β-cyclase gene located on chromosome 6 is meant by the “lycopene β-cyclase gene” in the present invention.

There is no particular limitation on the null allele of the lycopene β-cyclase gene as long as it results from a mutation in the lycopene β-cyclase gene and has thereby lost the function of the lycopene β-cyclase gene. That is, as long as the lycopene β-cyclase gene contains a mutation that prevents the lycopene β-cyclase gene from exhibiting the function of lycopene β-cyclase to convert lycopene to β-carotene, the mode of the mutation is not particularly limited. Examples of the null allele of the lycopene β-cyclase gene include null alleles known as the og gene and the ogc gene. Among these, the ogc gene is preferable as a null allele of the lycopene β-cyclase gene. The structure of the ogc gene is reported in the aforementioned Non-patent Literature 2.

Whether a target individual is homozygous for the null allele of the lycopene β-cyclase gene can be easily determined by methods known in this technical field, based on the nucleotide sequence information of the null allele of the lycopene β-cyclase gene. Alternatively, the determination can be made based on the phenotype of the tomato plant.

### Low-Fruit-pH Locus

The tomato plant in the present aspect further has the low-fruit-pH locus on chromosome 6. The “low-fruit-pH locus” is a quantitative trait locus that exhibits a phenotype of low fruit pH. Therefore, when a tomato plant has the locus, the tomato plant produces fruit that exhibits a low pH. Hereinafter, unless otherwise specified, pH refers to fruit pH. Details of the fruit pH will be described later.

In the present specification, the term “fruit” refers to fruit in a fully ripe stage, when the ripening stage of fruit of a tomato plant is divided into four stages: (i) a mature-green stage at around the time when fruit enlargement growth has finished; (ii) a breaker stage in which red color begins to appear on the fruit surface; (iii) a pink stage in which a light red color spreads over the whole fruit; and (iv) a fully ripe stage in which red color spreads over the whole fruit. Determination of fruit in the fully ripe stage can be made by a generally well-known method. For example, Sol Genomics Network-Tomato expression Atlas (SGN-TEA) (https://tea.solgenomics.net/anatomy_viewer/microscopy/slm82_fruit) may be used as an index. According to SGN-TEA, classification is as follows (the terms in parentheses are the designations used in SGN-TEA).
Mature-green stage (Mature Green): The fruit is fully expanded, and its surface exhibits a completely green color.
Breaker stage (Breaker): There is a definite break in color from green to tannish-yellow, pink or red on not more than 10% of the fruit surface.
Pink stage (Pink): Not less than 30% and not more than 60% of the fruit surface, in the aggregate, shows pink or red color.
Fully ripe stage (Light Red and Red Ripe): Not less than 60% of the surface, in the aggregate, shows pinkish red or red.

The presence or absence of the low-fruit-pH locus on chromosome 6 can be determined by a DNA SNP marker that is a single nucleotide polymorphism in a nucleotide sequence set forth in SEQ ID NO: 1 or a nucleotide sequence set forth in SEQ ID NO: 2 and that is associated with the low-fruit-pH locus. Specifically, it can be determined that a tomato plant possesses the low-fruit-pH locus if the tomato plant has a base defined by a SNP marker selected from the group consisting of (a) and (b) below, or has both bases defined by the respective SNP markers of (a) and (b):
(a) a SNP marker that is C at a base corresponding to a base at position 51 in the nucleotide sequence set forth in SEQ ID NO: 1; and
(b) a SNP marker that is T at a base corresponding to a base at position 51 in the nucleotide sequence set forth in SEQ ID NO: 2.

These SNP markers associated with the low-fruit-pH locus on chromosome 6 are also referred to simply as “SNP markers” in the present specification.

The SNP marker of (a) is a base located at a physical distance of 45,149,597 on chromosome 6 of the tomato plant, and the SNP marker of (b) is a base located at a physical distance of 45,149,796 on chromosome 6 of the tomato plant. In the process of completing the present invention, the inventors of the present invention discovered that these SNP markers are strongly linked to the low-fruit-pH locus and therefore identified them as SNP markers associated with the low-fruit-pH locus. The physical distances and the nucleotide sequences set forth in SEQ ID NOs: 1 and 2 are based on the reference genome sequence information of Solanum lycopersicum (genome database SL3.0/ITAG3.10 (Sol Genomics Network, http://www.solgenomics.net/)).

“A base corresponding to a base at position 51 in a nucleotide sequence set forth in SEQ ID NO: X (X=1 or 2)” means a SNP marker that can be considered identical to the present SNP marker. Depending on the variety and line, a part of the nucleotide sequence other than the SNP marker portion may also differ. Therefore, the SNP marker may be a base at a position corresponding to the base at position 51 in the nucleotide sequence set forth in SEQ ID NO: 1, within a sequence having a sequence identity of not less than 90%, not less than 95%, not less than 96%, not less than 97%, not less than 98%, or not less than 99% with respect to the nucleotide sequence set forth in SEQ ID NO: 1. Similarly, the SNP marker may be a base at a position corresponding to the base at position 51 in the nucleotide sequence set forth in SEQ ID NO: 1, within a sequence having a sequence identity of not less than 90%, not less than 95%, not less than 96%, not less than 97%, not less than 98%, or not less than 99% with respect to the nucleotide sequence set forth in SEQ ID NO: 2.

The SNP marker can be detected by a known SNP analysis method. Examples of the SNP analysis method include an analysis method based on detecting a SNP in a PCR-amplified fragment of chromosomal DNA from a tomato plant used as a test subject.

The tomato plant in accordance with the present aspect may be heterozygous or homozygous for the low-fruit-pH locus. Regardless of whether the tomato plant is heterozygous or homozygous for the low-fruit-pH locus, the fruit of the tomato plant exhibits a lower pH as compared with a case in which the tomato plant does not have the low-fruit-pH locus.

In the tomato plant in accordance with the present aspect, the low-fruit-pH locus can be a low-fruit-pH locus present in the genome of a tomato plant identified by Accession No. FERM BP-22545, which will be described later.

### Further Locus Exhibiting Additional Function

The tomato plant in the present aspect may have, on the same chromosome 6, another locus that expresses an additional function. By possessing multiple useful genes on the single chromosome, that is, on the same chromosome derived from one parental line, the breeding of an even more superior variety becomes possible.

As one aspect, the tomato plant in the present aspect may further have a root-knot nematode resistance locus on chromosome 6. Root-knot nematodes are pests that parasitize the roots of tomato plants, and parasitized plants lead to poor growth, death, etc. A variety having a root-knot nematode resistance gene (Mi) exhibits resistance to root-knot nematodes. The root-knot nematode resistance gene (Mi) has been reported to be located on chromosome 6 (Non-patent Literature: Gilbert, (1958) Tomato, Genet. Coop. Rep. 8:15-17; Patent Literature: Japanese Patent Application Publication, Tokukai, No. 2011-041569).

Whether or not the root-knot nematode resistance locus is present on chromosome 6 can be easily determined using a known marker. The known marker can be, for example, a marker disclosed in Garcia, B. E. et al., (2007) “A co-dominant SCAR marker, Mi23, for detection of the Mi-1.2 gene for resistance to root-knot nematode in tomato germplasm.” (http://invirlab.plantpath.wisc.edu/GeminivirusResistantTomatoes/Markers/MAS-Protocols/Mi23-SCAR.pdf).

### Fruit of Tomato Plant

The fruit of the tomato plant in the present aspect has a high lycopene content, similarly to known tomato plant lines having a high lycopene content, and also has a low pH.

The fruit pH of the tomato plant in the present aspect can be lower than that of known tomato plant lines with a high lycopene content (e.g., a known line possessing the ogc gene), and can be, for example, not more than 4.3. The fruit pH is an average of measured values for multiple fruits per line, and can typically be an average of measured values for not less than 10 fruits, for example, not less than 15 fruits. Furthermore, the fruit pH can be an average of measured values for not less than 10 fruits, for example, not less than 15 fruits (3 plant individuals x not less than 5 fruits), collected from one or more plant individuals per line. When using the tomato plant as a processed product, it is considered desirable for the pH to be not more than 4.3 for the purpose of suppressing microbial growth. As such, the tomato plant can be suitably used for processed products if the tomato plant has a pH of not more than 4.3. As a further aspect, the pH can be not more than 4.30, and further can be not more than 4.29, not more than 4.28, not more than 4.27, not more than 4.26, not more than 4.25, not more than 4.24, not more than 4.23, not more than 4.22, not more than 4.21, not more than 4.20, or not more than 4.19. The pH may be measured by a method commonly used in the technical field, and can be measured, for example, using a pH meter (manufactured by HORIBA, Ltd.).

The tomato fruit according to the present aspect can be provided for raw consumption or as a processed product. Examples of the processed product include tomato juice, tomato puree, tomato paste, and tomato ketchup.

The tomato plant in the present aspect includes, as an example, a tomato plant deposited under Accession No. FERM BP-22545 or a progeny line thereof. The deposit information is shown below.
Type of deposit: Seed
Depositary Institution: Room 120, 2-5-8 Kazusa-Kamatari, Kisarazu-shi, Chiba, Japan; National Institute of Technology and Evaluation (NITE), NITE Patent Microorganisms Depositary (NPMD)
Accession No. FERM BP-22545
Date of original deposit: Mar. 18, 2026.

### Method for Producing Tomato Plant

Next, one aspect of a method for producing the above-described tomato plant will be described. The method for producing a tomato plant that is one aspect of the present invention includes a crossing step of crossing a tomato plant having the null allele of the lycopene β-cyclase gene with a tomato plant having the low-fruit-pH locus, and a selection step of selecting, from a tomato plant obtained by the crossing step, a tomato plant that is homozygous for the null allele of the lycopene β-cyclase gene and is heterozygous or homozygous for the low-fruit-pH locus.

### Crossing Step

The tomato plant having the null allele of the lycopene β-cyclase gene used as a parental line is not particularly limited, and may be heterozygous or homozygous for the null allele of the lycopene β-cyclase gene. Examples of the tomato plant having the null allele of the lycopene β-cyclase gene include a tomato plant having the og gene, the ogc gene, or the like, which are known as mutants of the lycopene β-cyclase gene. Alternatively, the tomato plant having the null allele of the lycopene β-cyclase gene may be a mutant individual newly created by introducing, with use of a known mutagenesis technique, a mutation that causes a loss of function of the lycopene β-cyclase gene. The tomato plant having the null allele of the lycopene β-cyclase gene is preferably a tomato plant having the ogc gene, and more preferably a tomato plant that is homozygous for the ogc gene. Typically, the tomato plant having the null allele of the lycopene β-cyclase gene used as a parental line is a tomato plant that does not have the low-fruit-pH locus on the same chromosome 6.

The tomato plant having the low-fruit-pH locus used as a parental line may be any tomato plant having a base defined by the above-described SNP marker associated with the low-fruit-pH locus on chromosome 6. The low-fruit-pH locus in the tomato plant used as a parental line may be either heterozygous or homozygous. Typically, the tomato plant having the low-fruit-pH locus on chromosome 6 used as a parental line is a tomato plant that does not have the null allele of the lycopene β-cyclase gene on the same chromosome 6.

The crossing step in the present aspect may include not only producing an F1 individual obtained by crossing the tomato plant having the null allele of the lycopene β-cyclase gene and the tomato plant having the low-fruit-pH locus, but also producing an F2 individual obtained by self-pollination of the F1 individual or by crossing F1 individuals with each other.

### Selection Step

From a tomato plant obtained by the crossing step or a progeny line thereof (also simply referred to as “tomato plant obtained by crossing”), a tomato plant is selected that is homozygous for the null allele of the lycopene β-cyclase gene and is heterozygous or homozygous for the low-fruit-pH locus. The determination of whether a target individual is homozygous for the null allele of the lycopene β-cyclase gene and the determination of whether the target individual is heterozygous or homozygous for the low-fruit-pH locus may be made on the basis of nucleotide sequence information or based on phenotype.

In the above selection, when making a determination regarding the lycopene β-cyclase gene on the basis of nucleotide sequence information, the determination can be made by examining, with use of a known analysis method such as sequence analysis, whether or not the target individual is homozygous for the same null allele of the lycopene β-cyclase gene as the one in the parental line used in the crossing step.

In the above selection, when making a determination regarding the low-fruit-pH locus on the basis of nucleotide sequence information, the determination can be made by a method of detecting the above-described SNP marker associated with the low-fruit-pH locus. The method of detecting the SNP marker is not particularly limited and can be achieved by examination using any method known to a person skilled in the art. Examples of such a method include the cleaved amplified polymorphic sequence (CAPS) method, the sequencing method, the amplified fragment length polymorphism (AFLP) method, the restriction fragment length polymorphism (RFLP) method, and the TaqMan SNP genotyping assay. Among these, the CAPS method is preferable because it allows for determination with a simple operation.

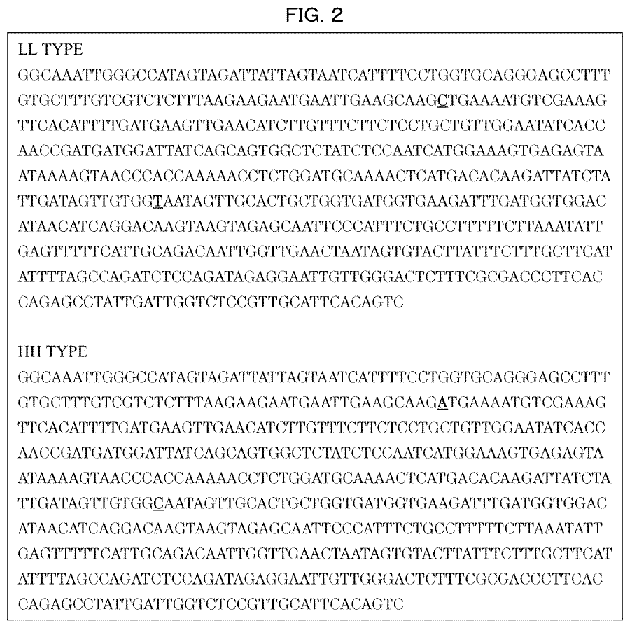

In the above selection, the determination regarding the lycopene β-cyclase gene can also be made based on phenotype. As an example, it is known that the flower color of a tomato plant that is homozygous for the null allele of the lycopene β-cyclase gene is orange, while the flower color of a tomato plant that is not homozygous for the null allele of the lycopene β-cyclase gene is yellow (see, for example, Non-patent Literature 2, FIG. 2). Therefore, if the flower color of the tomato plant obtained by crossing is orange, it can be presumed that the tomato plant is homozygous for the null allele of the lycopene β-cyclase gene.

In the above selection, the determination regarding the low-fruit-pH locus can also be made based on phenotype. As a non-limiting example, the determination can be made by measuring the fruit pH of the tomato plant obtained by crossing. The tomato plant obtained by the crossing step or of a progeny line thereof, having a fruit pH of not more than 4.30, is highly likely to have the low-fruit-pH locus. It is also possible to use indicators generally used in variety selection; for example, a target individual falling within the top 15% for low pH in a normal distribution based on fruit pH value data may be determined to be a plant individual having the low-fruit-pH locus.

As described above, as a result of prior investigation, it was confirmed that tomato plants homozygous for the ogc gene generally have a high lycopene content and a high fruit pH. Since the ogc gene is located on chromosome 6, this suggested that the ogc gene might be linked to a gene contributing to high fruit pH. Therefore, it was desirable to separate the undesirable trait (high pH) linked to the useful ogc gene. Accordingly, by using the SNP marker of the present disclosure, it has become possible to separate the undesirable trait (high pH) from the useful trait of high lycopene content.

### Method for Imparting Property of Low Fruit pH

A method for imparting a property of low fruit pH to a tomato plant that produces fruit having a high lycopene content is also included within the scope of the present invention. The method includes a crossing step of crossing a tomato plant having the null allele of the lycopene β-cyclase gene on chromosome 6 with a tomato plant having the low-fruit-pH locus on chromosome 6, and a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus. The crossing step and the selection step in the method can be the same as the crossing step and the selection step included in the above-described method for producing a tomato plant, respectively, and therefore, their description is omitted.

According to the present method, it is possible to impart a trait of low fruit pH, which is suitable for use in processed foods, to a tomato plant having the excellent trait of producing fruit having a high lycopene content.

### Use of SNP Marker

The use of a SNP marker for detecting a low-fruit-pH locus that results in low-pH tomato fruit is also included within the scope of the present invention. The use includes detecting at least one SNP marker selected from the group consisting of (a) and (b) below:
(a) a SNP marker that is C at a base corresponding to the base at position 51 in the nucleotide sequence set forth in SEQ ID NO: 1; and
(b) a SNP marker that is T at a base corresponding to the base at position 51 in the nucleotide sequence set forth in SEQ ID NO: 2.

The use of the marker makes it easy to breed a new tomato plant that has not only a trait related to lycopene content but also other excellent traits and the trait of low fruit pH. The method of using (detecting) the marker can be the same as the method described in the above-described method for producing a tomato plant, and therefore the description thereof is omitted.

The present invention is not limited to the embodiments, but can be altered by a skilled person in the art within the scope of the claims. The present invention also encompasses, in its technical scope, any embodiment derived by combining technical means disclosed in differing embodiments.

Aspects of the present invention can also be expressed as follows:

With the above embodiments considered together, the present invention can be summarized as follows.
<1> A tomato (Solanum lycopersicum) plant that is homozygous for a null allele of a lycopene β-cyclase gene on chromosome 6 and is heterozygous or homozygous for a low-fruit-pH locus on chromosome 6, the low-fruit-pH locus being identified by at least one SNP marker selected from the group consisting of (a) and (b) below:
   (a) a SNP marker that is C at a base corresponding to a base at position 51 in a nucleotide sequence set forth in SEQ ID NO: 1; and
   (b) a SNP marker that is T at a base corresponding to a base at position 51 in a nucleotide sequence set forth in SEQ ID NO: 2.
<2> The tomato plant as set forth in <1>, wherein fruit of the tomato plant has a pH of not more than 4.3.
<3> The tomato plant as set forth in <1> or <2>, wherein the low-fruit-pH locus is a low-fruit-pH locus present in the genome of a tomato plant identified by Accession No. FERM BP-22545.
<4> The tomato plant as set forth in any one of <1> to <3>, wherein the null allele of the lycopene β-cyclase gene is an ogc gene.
<5> The tomato plant as set forth in any one of <1> to <4>, further including a root-knot nematode resistance locus on chromosome 6.
<6> The tomato plant as set forth in any one of <1> to <5>, wherein the tomato plant is a tomato plant identified by Accession No. FERM BP-22545 or a progeny line thereof.
<7> The tomato plant as set forth in any one of <1> to <6>, wherein the tomato plant is a plant body or a part of a plant body.
<8> A method for producing a tomato plant as set forth in any one of <1> to <7>, the method including a crossing step of crossing a tomato plant having the null allele of the lycopene β-cyclase gene with a tomato plant having the low-fruit-pH locus; and a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus.
<9> The method for producing a tomato plant as set forth in <8>, wherein the selection step includes performing detection of the at least one SNP marker selected from the group consisting of (a) and (b).
<10> A method for imparting a property of low fruit pH to a tomato plant (Solanum lycopersicum) that produces fruit having a high lycopene content, the method including a crossing step of crossing a tomato plant having a null allele of a lycopene β-cyclase gene on chromosome 6 with a tomato plant having a low-fruit-pH locus on chromosome 6; and a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus.
<11> The method as set forth in <10>, wherein the selection step includes performing detection of at least one SNP marker selected from the group consisting of (a) and (b) below:
   (a) a SNP marker that is C at a base corresponding to a base at position 51 in a nucleotide sequence set forth in SEQ ID NO: 1; and
   (b) a SNP marker that is T at a base corresponding to a base at position 51 in a nucleotide sequence set forth in SEQ ID NO: 2.
<12> Use of a SNP marker for detecting a low-fruit-pH locus that results in low-pH tomato fruit, wherein the use includes detecting at least one SNP marker selected from the group consisting of (a) and (b) below:
   (a) a SNP marker that is C at a base corresponding to a base at position 51 in a nucleotide sequence set forth in SEQ ID NO: 1; and
   (b) a SNP marker that is T at a base corresponding to a base at position 51 in a nucleotide sequence set forth in SEQ ID NO: 2.

### EXAMPLES

### Example 1. Phenotype of Fruit pH Linked to Ogc Gene

Using a fixed tomato plant variety that does not possess the ogc gene and a fixed tomato plant variety that does possess the ogc gene, the relationship between the genotype of the ogc gene and the pH phenotype was investigated.

### Experiment 1. Measurement of Fruit pH

Fully ripe fruits were harvested from 30 arbitrarily selected fixed variety lines not possessing the ogc gene and 25 arbitrarily selected fixed variety lines possessing the ogc gene, among genetic resources held by Kagome Co., Ltd. The fruits were crushed in a mixer for 1 minute, then filled into sample bottles and heat-sterilized in boiling water for 30 minutes to be used as samples for the present experiment. After the sterilized samples were cooled to 20 °C, the pH of each sample was measured with a pH meter (manufactured by HORIBA, Ltd.). Table 1 shows a comparison of the fruit pH of the fixed varieties not possessing the ogc gene and the fruit pH of the fixed varieties possessing the ogc gene. The pH represents an average value for the 30 fixed variety lines not possessing the ogc gene and for the 25 fixed variety lines possessing the ogc gene. This test confirmed that the fixed varieties possessing the ogc gene have a higher fruit pH compared to the fixed varieties not possessing the ogc gene.

TABLE 1

| Line name | pH | Standard deviation |
|---|---|---|
| Lines not posessing ogc gene | 4.35 | 0.022 |
| Lines posessing ogc gene | 4.46 | 0.023 |

### Example 2. Preparation of pH-Related SNP Markers and Analysis of Linkage with Ogc Gene

### Experiment 2-1. Preparation of pH-Related SNP Markers

With use of the genetic resources held by Kagome Co., Ltd., a DNA marker linked to pH phenotype was selected from among DNA markers having polymorphisms on chromosome 6 in varieties differing in pH phenotype. As a result, two SNPs linked to pH phenotype were found and were designated as pH-related SNP markers (markers 1 and 2), respectively. The information on the SNP markers is as follows.

TABLE 2

| | | Low-pH | High-pH |
|---|---|---|---|
| | Physical distance of SNP | type SNP | type SNP |
| Marker 1 | 45,149,597 | C | A |
| Marker 2 | 45,149,796 | T | C |

In Table 2, “Physical distance of SNP” refers to the position of each SNP on chromosome 6. The SNPs are at the base at position 51 of the sequences represented by SEQ ID NOs: 1 and 2, respectively, and each of the bases varies depending on the fruit pH phenotype. In the following Examples, a case where a plant individual has a homozygous genotype of a low-pH type for (is homozygous for a low-pH type SNP at) both of the pH-related SNP markers 1 and 2 is defined as LL type; a case where a plant individual has a homozygous genotype of a high-pH type for (is homozygous for a high-pH type SNP at) both of the pH-related SNP markers 1 and 2 is defined as HH type; and a case where a plant individual has a heterozygous genotype of low-pH and high-pH types for both of the pH-related SNP markers 1 and 2 (i.e., a high-pH type SNP occurs at both of one pair of pH-related SNP markers 1 and 2, and a low-pH type SNP occurs at both of the other pair of pH-related SNP markers 1 and 2) is defined as LH type.

### Experiment 2-2. Linkage of Ogc Gene and Low-Fruit-pH Locus

Six lines possessing ogc among the genetic resources held by Kagome Co., Ltd. were checked for the genotypes of the above-described PH-related SNP markers. The genotypes were found to be HH type for all of the six lines.

The genotypes of the pH-related SNP markers were determined by the CAPS method. The PCR conditions for obtaining a PCR-amplified fragment used for CAPS were as follows:

### Primer Set:

Forward primer: ggcaaattgggccatagtag (SEQ ID NO: 3)
Reverse primer: gactgtgaatgcaacggaga (SEQ ID NO: 4)

### Amplification Conditions:

Annealing temperature: 56 °C, cycles: 30 to 40, extension time: 1 minute.

FIG. 2 shows the nucleotide sequences of PCR-amplified fragments, each containing bases of the pH-related SNP markers, for the LL type and the HH type, respectively (LL type: SEQ ID NO: 5, HH type: SEQ ID NO: 6). The bold letters indicate polymorphic bases of the pH-related SNP markers.

When an obtained PCR-amplified fragment is treated with a restriction enzyme AluI, cleavage of the PCR-amplified fragment occurs if a low-pH type SNP is present at the pH-related SNP marker 1. Therefore, PCR-amplified fragments derived from an LL type plant individual are cleaved, but PCR-amplified fragments derived from an HH type plant individual are not cleaved. PCR-amplified fragments derived from an LH type plant individual result in a mixture of cleaved amplified fragments and uncleaved amplified fragments.

### Example 3. Analysis of Genotypes and Phenotypes Regarding pH-Related Gene

In the present experiment, line PK461, which has a low pH phenotype, and line PK367, which has a high pH phenotype, were used from the genetic resources held by Kagome Co., Ltd. PK461 and PK367 were crossed to obtain an F1 individual. The F1 individual was self-pollinated to obtain F2 individuals. The obtained population of F2 individuals was checked for the genotypes of the pH-related SNP markers. The genotypes were determined by CAPS under the same conditions as in Experiment 2-2. PK461 and PK367 were also checked for the genotypes of the pH-related SNP markers in a similar fashion. Among the F2 population, an LL-type homozygous plant individual was designated as F2-1, an LH-type heterozygous plant individual as F2-2, and an HH-type homozygous plant individual as F2-3. Table 3 shows the genotypes of the pH-related SNP markers of the parental lines used for crossing and the lines obtained by crossing.

TABLE 3

| Line name | pH marker |
|---|---|
| PK461 | LL |
| PK367 | HH |
| F2-1 | LL |
| F2-2 | LH |
| F2-3 | HH |

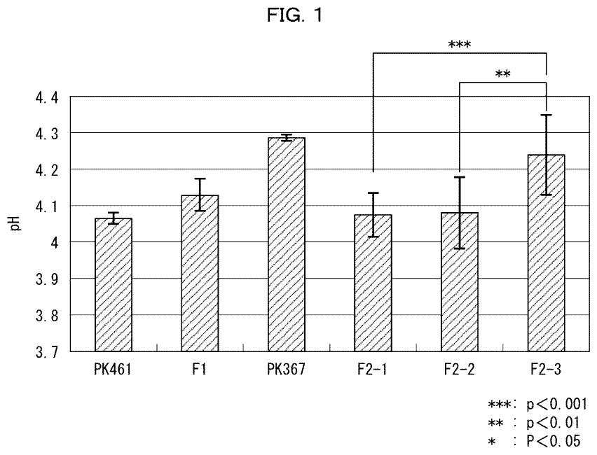

The fruit pH of these lines was measured. In the fruit pH measurement, the pH of 5 fruits from each of three plant individuals of each line was measured. The pH measurement was repeated three times in total, and an average value of 45 fruits (3 plant individuals x 5 fruits x 3 repetitions) was taken as a measurement result for that line. To examine the effect of repetition on the obtained measurement data, a multi-way analysis of variance was performed to analyze the presence or absence of the effect of repetition. The results showed no significant difference between trials, indicating no effect of repetition. The measurement results are shown in FIG. 1. Note that the genotype of the F1 individual at the pH-related SNP markers was not confirmed, but is theoretically the LH type. As shown in FIG. 1, plant individuals that were of the LL type (F2-1) at the pH-related SNP markers and plant individuals that were of the LH type (F2-2) at the pH-related SNP markers had a pH significantly lower than that of plant individuals that were of the HH type (F2-3).

### Example 4. Breeding Example 1 of Line in which High-pH Trait was Separated

From the genetic resources held by Kagome Co., Ltd., line PK886 (LL type) and line PK431 (HH type and homozygous for ogc gene) were used as parental lines and crossed to obtain an F1 individual. After obtaining F2 individuals by self-pollination of the F1 individual, the F2 individuals were further self-pollinated to obtain a population of 400 tomato plants. From the obtained 400 tomato plants, one tomato plant (Kc13-13-118) that was of the LL type and homozygous for the ogc gene was obtained by selection through sequence analysis for the ogc gene and CAPS for the pH-related SNP markers. The fruit pH of tomato plants of a self-pollinated progeny (Kc13-13-118-1) of Kc13-13-118 was measured with a pH meter (manufactured by HORIBA, Ltd.) to confirm the pH phenotype. As a result, Kc13-13-118-1 showed a lower pH value compared to the parental line PK431. The results are shown in Table 4. It was shown that by using the pH-related SNP markers, it is possible to select a tomato plant that has a low fruit pH while being homozygous for the ogc gene.

TABLE 4

| Line name | pH marker | ogc gene | pH |
|---|---|---|---|
| Kc13-13-118-1 | LL | Homozygous | 4.12 |
| PK431 | HH | Homozygous | 4.40 |

### Example 5. Breeding Example 2 of Line in which High-pH Trait was Separated

From the genetic resources held by Kagome Co., Ltd., line PK894 (LL type) and line PK908 (HH type and homozygous for ogc gene) were used as parental lines and crossed to obtain F1. After obtaining F2 by self-pollination of F1, F2 was further self-pollinated to obtain a population of 200 tomato plants. From the obtained 200 tomato plants, 5 tomato plants (Kc12-380-1-1, Kc12-380-1-2, Kc12-380-1-3, Kc12-380-1-4, and Kc12-380-1-5) each of which was of the LL type and heterozygous for the ogc gene were obtained by selection through sequence analysis for the ogc gene and CAPS for the pH-related SNP markers. From among an F4 generation obtained by self-pollination of these tomato plants, tomato plants homozygous for the ogc gene (Kc12-380-1-1-1, Kc12-380-1-2-1, Kc12-380-1-3-1, Kc12-380-1-4-1, Kc12-380-1-5-1) were checked for the fruit pH phenotype. The results are shown in Table 5. It was shown that even when tomato plants with a genetic background different from that of Example 4 are used as parental lines, by using the pH-related SNP markers, it is possible to select a tomato plant that has a low fruit pH while being homozygous for the ogc gene.

TABLE 5

| Line name | pH marker | ogc gene | pH |
|---|---|---|---|
| PK894 | LL | Not possessing | 4.18 |
| PK908 | HH | Homozygous | 4.32 |
| Kc12-380-1-1-1 | LL | Homozygous | 4.19 |
| Kc12-380-1-2-1 | LL | Homozygous | 4.21 |
| Kc12-380-1-3-1 | LL | Homozygous | 4.27 |
| Kc12-380-1-4-1 | LL | Homozygous | 4.22 |
| Kc12-380-1-5-1 | LL | Homozygous | 4.26 |

### Example 6. Selection of Tomato Plants Possessing Root-Knot Nematode Resistance Locus

For the five tomato plants Kc12-380-1-1-1 through Kc12-380-1-5-1 obtained in Example 5, whether a root-knot nematode resistance gene (Mi gene) was present on chromosome 6 was examined. As a result, it was found that the lines Kc12-380-1-1-1 and Kc12-380-1-3-1 possessed the Mi gene. Note that the presence of the Mi gene was confirmed by sequence analysis using the aforementioned known marker disclosed by Garcia et al. The PCR conditions for obtaining a PCR-amplified fragment for the sequence analysis were as follows:

### Primer Set:

Forward primer: tggaaaaatgttgaatttcttttg (SEQ ID NO: 7)
Reverse primer: gcatactatatggcttgtttaccc (SEQ ID NO: 8)

### Amplification Conditions:

Annealing temperature: 54 °C, cycles: 40, extension time: 1 minute.

The present invention can be utilized in a food industry that uses the fruit of tomato plants.

1. A tomato (Solanum lycopersicum) plant that is homozygous for a null allele of a lycopene β-cyclase gene on chromosome 6 and is heterozygous or homozygous for a low-fruit-pH locus on chromosome 6, the low-fruit-pH locus being identified by at least one SNP marker selected from the group consisting of (a) and (b) below:
(a) a SNP marker that is C at a base corresponding to a base at position 51 in a nucleotide sequence set forth in SEQ ID NO: 1; and
(b) a SNP marker that is T at a base corresponding to a base at position 51 in a nucleotide sequence set forth in SEQ ID NO: 2.

2. The tomato plant of claim 1, wherein fruit of the tomato plant has a pH of not more than 4.3.

3. The tomato plant of claim 1, wherein the low-fruit-pH locus is a low-fruit-pH locus present in the genome of a tomato plant identified by Accession No. FERM BP-22545.

4. The tomato plant of claim 1, wherein the null allele of the lycopene β-cyclase gene is an old-gold-crimson(ogc) gene.

5. The tomato plant of claim 1, further comprising a root-knot nematode resistance locus on chromosome 6.

6. The tomato plant of claim 1, wherein the tomato plant is a tomato plant identified by Accession No. FERM BP-22545 or a progeny line thereof.

7. The tomato plant of claim 1, wherein the tomato plant is a plant body or a part of a plant body.

8. A fruit of the tomato plant of claim 1.

9. A method for producing the tomato plant of claim 1, the method comprising:
a crossing step of crossing a tomato plant having the null allele of the lycopene β-cyclase gene with a tomato plant having the low-fruit-pH locus; and
a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus.

10. The method of claim 9, wherein the selection step includes performing detection of the at least one SNP marker selected from the group consisting of (a) and (b).

11. A method for imparting a property of low fruit pH to a tomato plant (Solanum lycopersicum) that produces fruit having a high lycopene content, the method comprising:
a crossing step of crossing a tomato plant having a null allele of a lycopene β-cyclase gene on chromosome 6 with a tomato plant having a low-fruit-pH locus on chromosome 6; and
a selection step of selecting, from a tomato plant obtained by the crossing step or a progeny line thereof, a tomato plant that is homozygous for the null allele of the lycopene β-cyclase gene and is heterozygous or homozygous for the low-fruit-pH locus.

12. The method of claim 11, wherein the selection step includes performing detection of at least one SNP marker selected from the group consisting of (a) and (b) below:
(a) a SNP marker that is C at a base corresponding to a base at position 51 in a nucleotide sequence set forth in SEQ ID NO: 1; and
(b) a SNP marker that is T at a base corresponding to a base at position 51 in a nucleotide sequence set forth in SEQ ID NO: 2.

13. A method for detecting a low-fruit-pH locus that results in low-pH tomato fruit comprising detecting at least one SNP marker selected from the group consisting of (a) and (b) below:
(a) a SNP marker that is C at a base corresponding to a base at position 51 in a nucleotide sequence set forth in SEQ ID NO: 1; and
(b) a SNP marker that is T at a base corresponding to a base at position 51 in a nucleotide sequence set forth in SEQ ID NO: 2.


SEQUENCE LISTING (8 sequences)

SEQ ID NO: 1 - DNA, 101 bp, Solanum lycopersicum
  source 1..101: mol_type = genomic DNA
gggagcctttgtgctttgtcgtctctttaagaagaatgaattgaagcaagctgaaaatgtcgaaagttcacattttgatgaagttgaacatcttgtttctt

SEQ ID NO: 2 - DNA, 101 bp, Solanum lycopersicum
  source 1..101: mol_type = genomic DNA
acctctggatgcaaaactcatgacacaagattatctattgatagttgtggtaatagttgcactgctggtgatggtgaagatttgatggtggacataacatc

SEQ ID NO: 3 - DNA, 20 bp, synthetic construct
  source 1..20: mol_type = other DNA
ggcaaattgggccatagtag

SEQ ID NO: 4 - DNA, 20 bp, synthetic construct
  source 1..20: mol_type = other DNA
gactgtgaatgcaacggaga

SEQ ID NO: 5 - DNA, 547 bp, Solanum lycopersicum
  source 1..547: mol_type = genomic DNA
ggcaaattgggccatagtagattattagtaatcattttcctggtgcagggagcctttgtgctttgtcgtctctttaagaagaatgaattgaagcaagctgaaaatgtcgaaagttcacattttgatgaagttgaacatcttgtttcttctcctgctgttggaatatcaccaaccgatgatggattatcagcagtggctctatctccaatcatggaaagtgagagtaataaaagtaacccaccaaaaacctctggatgcaaaactcatgacacaagattatctattgatagttgtggtaatagttgcactgctggtgatggtgaagatttgatggtggacataacatcaggacaagtaagtagagcaattcccatttctgcctttttcttaaatattgagtttttcattgcagacaattggttgaactaatagtgtacttatttctttgcttcatattttagccagatctccagatagaggaattgttgggactctttcgcgacccttcaccagagcctattgattggtctccgttgcattcacagtc

SEQ ID NO: 6 - DNA, 547 bp, Solanum lycopersicum
  source 1..547: mol_type = genomic DNA
ggcaaattgggccatagtagattattagtaatcattttcctggtgcagggagcctttgtgctttgtcgtctctttaagaagaatgaattgaagcaagatgaaaatgtcgaaagttcacattttgatgaagttgaacatcttgtttcttctcctgctgttggaatatcaccaaccgatgatggattatcagcagtggctctatctccaatcatggaaagtgagagtaataaaagtaacccaccaaaaacctctggatgcaaaactcatgacacaagattatctattgatagttgtggcaatagttgcactgctggtgatggtgaagatttgatggtggacataacatcaggacaagtaagtagagcaattcccatttctgcctttttcttaaatattgagtttttcattgcagacaattggttgaactaatagtgtacttatttctttgcttcatattttagccagatctccagatagaggaattgttgggactctttcgcgacccttcaccagagcctattgattggtctccgttgcattcacagtc

SEQ ID NO: 7 - DNA, 24 bp, synthetic construct
  source 1..24: mol_type = other DNA
tggaaaaatgttgaatttcttttg

SEQ ID NO: 8 - DNA, 24 bp, synthetic construct
  source 1..24: mol_type = other DNA
gcatactatatggcttgtttaccc